(19) United States
(12) Patent Application Publication
(10) Pub. No.: US 2003/0236285 A1
(43) Pub. Date: Dec. 25, 2003

(54) Stabilized pharmaceutical compositions containing benzimidazole compounds

(76) Inventors: Mahendra R. Patel, East Brunswick, NJ (US); Amol Singh Matharu, Bedminster, NJ (US); Chuanbin Wu, Franklin Park, NJ (US); Ashish Anilbhai Patel, Kendall Park, NJ (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/431,271

(22) Filed: May 7, 2003

Related U.S. Application Data
(63) Continuation-in-part of application No. 10/164,744, filed on Jun. 7, 2002.

(51) Int. Cl. A61K 31/4439; A61K 31/7016; A61K 9/24

(52) U.S. Cl. 514/338; 514/57; 424/471

(57) ABSTRACT

An oral pharmaceutical composition in a solid dosage form comprising:

a) a single core comprising a proton pump inhibitor and a lubricant, wherein said single core has an exterior surface;

b) an enteric compression coating comprising a polymer and a lubricant, wherein said enteric compression coating is on the exterior surface of said single core, without a separating layer between said single core and said enteric compression coating; and

c) optionally, a polymer overcoating on said enteric compression coating.

### FIELD OF THE INVENTION

The present invention relates to new, stabilized compositions containing proton-pump inhibitors (PPI) from the benzimidazole class of compounds.

### BACKGROUND OF THE INVENTION

Certain benzimidazoles are anti-ulcerous compounds known for decreasing gastric acid secretion. However, these compounds, also known as PPI, are susceptible to degradation/transformation in acidic reacting and neutral media. The degradation is catalyzed by acidic reacting compounds and the PPIs are usually stabilized in mixtures with alkaline reacting compounds. In respect to the stability properties of the benzimidazole compounds mentioned above, it is obvious that those in an oral solid dosage form must be protected from contact with the acidic reacting gastric juice and the active substance must be transferred in intact form to that part of the gastrointestinal tract where pH is less acidic, neutral or alkaline and where rapid absorption of the pharmaceutically active substance, i.e., the benzimidazole derivative, can occur.

U.S. Pat. No. 4,853,230 has shown that a pharmaceutical dosage form of these benzimidazole derivatives can be protected from contact with acidic gastric juice by an enteric coating layer. Such preparations contain an alkaline core material comprising the active substance, a separating layer and an enteric coating layer. Ordinary enteric coating layers, however, comprise compounds which contain acidic groups. If covered with such an enteric coating layer, the acid labile substance may rapidly decompose by direct or indirect contact with the acidic groups resulting in discoloration of the content and loss in content of the active compound with the passage of time. The discoloration can be avoided by applying-some type of separating layer between the core material comprising the susceptible these benzimidazole derivatives and the enteric coating layer.

U.S. Pat. Nos. 4,628,098; 4,853,230; 4,026,560; 5,689,333; 5,045,321; 5,093,132; 5,433,959; and 6,013,281 teach various stabilizing agents for these benzimidazole derivatives in the core tablets. These references also show that such compounds are stable in the presence of basic inorganic salts of magnesium, calcium, potassium and sodium. The stability is further consolidated by separating the acidic components of the enteric coat by an intermediate coating, where the core material are pellets.

U.S. Pat. No. 6,013,281 also discloses that a separating layer is formed in situ by direct application of an acidic enteric material on to the alkaline core containing the PPI.

Tabata et al., “Stabilization of New Antiulcer Drug (lansoprazole) in the Solid Dosage Forms”, Drug. Dev. Ind. Pharm., Vol. 18, pp. 1437-1447 (1992) showed that the rate of degradation of lansoprazole, a representative of the benzimidazole series, was reduced to negligible in pH higher than 7.

WO 98/00115 teaches the use of aqueous application of partially neutralized enteric polymer applied directly onto the reactive core. Similar application was disclosed in U.S. Pat. No. 5,225,202.

The need to use a separating layer requires the application of two separate functional coating operations which increases the length of the manufacturing process and the cost of the product. It would desirable to provide an alternative oral dosage composition containing a PPI, that does not rely upon the use of an intermediate or separating layer to stabilize the PPI contained therein.

### SUMMARY OF THE INVENTION

Applicants have developed an oral pharmaceutical composition in the form of a tablet that avoids the need to use a separating layer to separate the tablet core containing the PPI from the enteric coating layer in a tablet dosage form.

Thus, in one embodiment, the present invention is directed toward an oral pharmaceutical composition in a solid dosage form comprising:

a) a single core comprising a proton pump inhibitor and a lubricant, wherein said single core has an exterior surface;

b) an enteric compression coating comprising a polymer and a lubricant, wherein said enteric compression coating is on the exterior surface of said single core, without a separating layer between said single core and said enteric compression coating; and

c) optionally, a polymer overcoating on said enteric compression coating.

In another embodiment, the present invention is directed towards an oral pharmaceutical composition in a solid dosage form comprising:

a) a single core comprising a proton pump inhibitor, a disintegrant, a filler and a lubricant, wherein said single core has an exterior surface;

b) an enteric compression coating comprising a polymer and a lubricant, wherein said enteric compression coating is on the exterior surface of said single core, without a separating layer between said single core and said enteric compression coating; and

c) optionally, a polymer overcoating on said enteric compression coating

In another embodiment the present invention is directed toward an oral pharmaceutical composition in a solid dosage form comprising:

a) a single core comprising a proton pump inhibitor and a lubricant, said single core being essentially free of an alkaline reacting agent, wherein said single core has an exterior surface;

b) an enteric compression coating comprising a polymer and a lubricant, wherein said enteric compression coating is on the exterior surface of said single core, without a separating layer between said single core and said enteric compression coating; and

c) optionally, a polymer overcoating on said enteric compression coating.

In another embodiment, the present invention is directed towards a process for preparing an oral pharmaceutical composition in a solid dosage form comprising:

a) forming a single core comprising a proton pump inhibitor and a lubricant, wherein said single core has an exterior surface;

b) compression coating an enteric polymer comprising a proton pump inhibitor and a lubricant onto the exterior surface of said single core, in the absence of water and organic solvents, and without forming a separating layer between said single tablet core and said enteric coating; and

c) optionally, applying a polymer overcoating on said enteric compression coating.

The single tablet core may contain a PPI selected from the group consisting of rabeprazole, omeprazole, esomeprazole, lansoprazole, leminoprazole, pantoprazole or mixtures thereof.

The enteric coating may contain a polymer selected from cellulose acetate phthalate, hydroxypropylmethylcellulose acetate succinate (HPMCAS), hydroxypropylmethylcellulose phthalate (HPMCP), polyvinylacetate phthalate, carboxymethylethylcellulose, acrylic acid polymers and co-polymers and methacrylic acid polymers and co-polymers or combinations thereof.

The present invention has the advantage of providing an oral pharmaceutical composition containing a labile PPI in the form of a tablet that can provide improved stability of the PPI contained therein against degradation and/or discoloration by moisture and/or heating.

Another advantage of the present invention is that it provides an oral pharmaceutical composition containing a labile PPI in the form of a tablet whose design and/or construction is greatly simplified over other known tableted compositions.

Another advantage of the present invention is that it provides an oral pharmaceutical composition containing a labile PPI that allows control of the release rate of said labile PPI within wide margins.

Another advantage of the present invention is that it provides a process for preparing an oral pharmaceutical composition containing a labile PPI, that can eliminate the use of water or organic solvents during coating of the tablet core, i.e., can be solvent-free.

Another advantage of the present invention is that it provides a process for preparing an oral pharmaceutical composition containing a labile PPI that can eliminate the need for heating during process, i.e., can be processed at ambient temperatures.

Another advantage of the present invention is that it provides an oral pharmaceutical composition and a process for preparation thereof, containing a labile PPI in the form of a tablet that does not require a separating layer to separate the core unit containing the acid-labile PPI from the enteric coating.

Another advantage of the present invention is that it provides a process for preparing an oral pharmaceutical composition, containing a labile PPI in the form of a tablet that can prevent the in situ formation of a separating layer between the core unit containing the acid-labile PPI from the enteric coating.

Another advantage of the present invention is that it provides a process for preparing an oral pharmaceutical composition containing a labile PPI in the form of a tablet, that can be carried out or produced using conventional pharmaceutical equipment.

### DETAILED DESCRIPTION OF THE INVENTION

Utilization of an enteric material as a non-interactive dry application of an otherwise highly interactive material has not been discussed in the prior art, and has not been disclosed in any of the previous patents.

The PPI in an oral solid dosage form should be protected from contact with the acid reacting gastric juice and the active substance should be transferred in intact form to that part of the gastrointestinal tract where the pH is less acidic, neutral or alkaline and where rapid absorption of the pharmaceutically active substance can occur.

A) Core

The terms “tablet core”, “core”, “single core”, “core tablet”, “single tablet core” or “single tablet core unit” have the same meaning and can be used interchangeably. Also, the terms “benzimidazole”, “benzimidazole compound”, “proton pump inhibitor” and “PPI” have the same meaning and can be used interchangeably.

Suitable benzimidazole compounds that can be employed as an active ingredient in the composition of the present invention include those of formula (I)

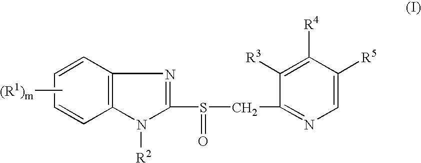

wherein

R¹ is hydrogen, alkyl, halogen, cyano, carboxy, carboalkoxy, carboalkoxyalkyl, carbamoyl, carbamoylalkyl, hydroxy, alkoxy, hydroxyalkyl, trifluoromethyl, acyl, carbamoyloxy, nitro, acyloxy, aryl, aryloxy, alkylthio or alkylsulfinyl;

R² is hydrogen, alkyl, acyl, carboalkoxy, carbamoyl, alkylcarbamoyl, dialkylcarbamoyl, alkylcarbonylmethyl, alkoxycarbonylmethyl or alkylsulfonyl;

R³ and R⁵ are the same or different and each can be hydrogen, alkyl, alkoxy or alkoxyalkoxy;

R⁴ is hydrogen, alkyl, alkoxy which may optionally be fluorinated, or alkoxyalkoxy; and m is an integer of 0 through 4.

Representative examples of such PPIs include rabeprazole, omeprazole, esomeprazole, pariprazole, lansoprazole, leminoprazole, pariprazole, pantoprazole or mixtures thereof.

The PPIs employed in the present invention may be used in neutral form or in the form of an alkaline metal salt, such as for instance, the salt of potassium, sodium, lithium, magnesium and/or calcium. Also, the benzimidazole compounds cited above may be used in a neutral form, in a racemic mixture, in the form of a substantially pure enantiomer thereof, as an alkaline salt of the racemic mixture or a single enantiomer, or combinations thereof. The amount of PPI can range from about 5% to about 75% by weight, from about 10% to about 70% by weight or from about 15% to about 60% by weight of the oral pharmaceutical composition. Alternatively, the oral pharmaceutical composition or tablet can contain a known mass of the PPI, such as 10, 15, 20, 30 or 40 mg.

The term “labile” refers to the property that the PPI are susceptible to degradation in the presence of acid and neutral media, humidity and/or elevated temperatures. For example, degradation of PPI can be catalyzed by acids or acid containing compounds. The PPI may also be unstable in the presence of water or high humidity.

Suitable inert fillers that can be used in the core include lactose, mannitol, starch, sucrose, glucose, hydroxypropyl cellulose, low-substituted hydroxypropyl cellulose, ethylcellulose, hydroxypropyl methylcellulose phthalate, diacetylated monoglycerides, talc, titanium dioxide and other excipients. The amount of filler can range from about 10% to about 90% by weight of the tablet.

Suitable disintegrants that can be used in the core can include sodium starch glycolate or sodium crosscarmellose. The amount of disintegrant can range from about 0.5% to about 30% by weight of the tablet.

Suitable lubricants that can be used in the core can include dry or solid lubricants, such as magnesium stearate, calcium stearate, sodium stearate, sodium stearyl fumarate and waxes, such as polyethylene glycol (solid form) and carnauba wax. The lubricant can be employed in both the enteric compression coating and in the core. The amount of lubricant can range from about 0.5 to about 30% by weight of each tablet or solid dosage form component, also from about 5 to about 25 percent by weight, also from about 10 to about 15 percent by weight. Alternatively, the amount of lubricant in the pharmaceutical composition can range from about 0.1% to about 10-20%, also about 0.2% to about 6% by weight. Alternatively, the amount of lubricant in the tablet can range from about 0.01 parts to about 1.5 parts by weight of the lubricant per one part PPI (about 0.01-1.5 parts lubricant:one part PPI).

The PPI is mixed with suitable pharmaceutical constituents, such as those described above for the fillers, disintegrants and lubricants and the resulting mixture is compressed into the core or tablet core unit. Moreover, the core tablet core of the present invention should be essentially free of alkaline reacting agents or compounds, such as those cited in U.S. Pat. No. 6,013,281. The PPI should not be seeded or layered prior to being compressed into the core unit. The size of the formulated core material is approximately between about one and about 20 mm and preferably between about 3 mm and about 15 mm. The manufactured core tablet containing the PPI can be covered with an enteric outer coating or layer. After preparation, the single core tablet has an exterior surface where the enteric outer coating is applied or coated.

B) Enteric Compression Coating or Layer

The terms “enteric coating”, “enteric compression coating”, “enteric compression layer”, “enteric outer coating”, “enteric layer” or “enteric outer layer” have the same meaning and can be used interchangeably. The enteric coating should be inert or substantially non-interacting with the single, tablet core containing the PPI. The enteric coating may contain ingredients, such as one or more polymers, release rate agents, lubricants, anti-tacking agents, colorants, pigments or other additives to obtain a tablet of good appearance. The amount of enteric coating in the tablet can range from about 0.1-0.4 parts to about 3 parts by weight of enteric coating per one part by weight tablet core (about 0.1-0.4 to 3 parts by weight enteric coating:one part tablet core). However, the enteric outer coating does not contain any PPI or other active drug ingredient.

Suitable polymers that can be used in the enteric coating can include anionic co-polymers based on methacrylic acid esters, commercially available as Eudragit L 100 and Eurdragit S 100, trademarks of Rohm, GmbH & Co., KG, Darmstadt, Germany. This enteric coating is insoluble below pH 5 and is thus resistant to gastric fluid. By salt formation in the neutral or weakly alkaline medium of the intestinal fluid, the enteric coating dissolves stepwise at pH values greater than 5.5-7.5. Another suitable polymer that can be used includes HPMCP or HPMCAS, commercially available from the Shin-Etsu Chemical Co. Ltd. A sole polymer can be employed such as HPMCAS or a mixture of polymers can be used, such as Eudragit and HPMCP. Thus, polymers can be cellulose acetate phthalate, HPMCAS, HPMCP, polyvinylacetate phthalate, carboxymethylethylcellulose, acrylic acid polymers and co-polymers and methacrylic acid polymers and co-polymers. The non-interacting property of such enteric coatings can be obtained or enhanced by neutralizing free acids in the enteric polymer with an inorganic or organic alkaline material, such as sodium hydroxide, magnesium hydroxide, meglumine and the like. The neutralized polymer results in enhanced stabilization of the tablet core. The amount of each polymer employed in the enteric coating can range from about 5% to about 99% by weight of the composition.

Suitable release rate agents that can be used in the enteric coating can include lactose, mannitol, starch, sucrose, glucose, hydroxypropylcellulose, low-substituted hydroxypropylcellulose, ethylcellulose, HPMCP, diacetylated monoglycerides, talc or titanium dioxide. The amounts of release agent employed in the enteric coating can range from about 0.5% to about 95% by weight of the composition.

Suitable lubricants that can be used in the core can include dry or solid lubricants, such as magnesium stearate, calcium stearate, silicon dioxide, or sodium stearate and waxes, such as carnauba wax. The lubricant can be employed in both the enteric compression coating and in the core or tablet core. The amount of lubricant in either the enteric coating layer or in the core can range from about 0.5 to about 30% by weight of each tablet or solid dosage form component, also from about 5 to about 25 percent by weight, also from about 10 to about 15 percent by weight. Alternatively, the amount of lubricant in the pharmaceutical composition can range from about 0.1% to about 10-20%, also about 0.2% to about 6% by weight. Alternatively, the amount of lubricant in the tablet can range from about 0.01 parts to about 1.5 parts by weight of the lubricant per one part PPI (about 0.01-1.5 parts lubricant:one part PPI).

The ingredients used in the enteric coating are dry or solid (with the exception of the optional polymer over-coating, discussed below) and can be blended or mixed together in the absence of water or organic solvents. The dry blend or mixture can be compressed (i.e., compression-coated) directly onto the exterior surface of the core or tablet core, using conventional procedures. One skilled in the art can utilize a range of compression forces or tablet hardnesses which provide the desired attributes for the enteric compression coating, such as acid protection or release in the post-stomach region. For example, one can prepare a series of tablet samples at different compression forces or tablet hardnesses and select a range which meets desired physical and performance attributes, such as tablet friability and dissolution profiles. These attributes are also described in the United States Pharmacopeia (USP) 26, United States Pharmacopeial Convention Inc., (2003) and Chapter 724 (drug release)-Delayed-Release (enteric-coated) articles-general drug release standards. For example, the range of hardnesses for a tablet with the enteric compression coating can range from about 4 SCU to about 30 SCU, also from about 7 to about 15 SCU. The compressive forces can range from about 0.1 ton to about 3 tons, also from about 0.3 to about 2 tons, also from about 0.5 ton to about 1.5 tons. Alternatively, the dry mixture can be sprayed or dispersed directly onto the core or tablet core and then compressed as described above. The dry blend or mixture that is compressed onto the exterior surface of the core or tablet core forms the enteric compression coating for the pharmaceutical composition. After the enteric compression coating is applied to the core or tablet core, there is no separating layer between the core and the enteric compression coating.

C. Optional Polymer Over-Coating

Tablets with the enteric coating are then covered with optionally one or more finishing polymer over-coating or tablet film coat(s) or layer(s) to obtain tablets of good appearance, smoothness, color or functionality, such as modified release. The maximum thickness of the applied over-coating layer(s) is normally limited by processing conditions and the desired dissolution or release profile. For example, the tablet film(s) can be a thin coat as compared to the enteric coating. The polymer over-coating can be water soluble or water soluble/swellable in water or have a solubility that is pH dependent. Further, the over-coating can be rapidly disintegrating or even insoluble in water. The materials for the over-coating layer can be pharmaceutically acceptable excipients, such as the same polymers used in the enteric coating layer, sugar, polyethylene glycol, polyvinylpyrrolidone, polyvinyl alcohol, polyvinyl acetate, hydroxypropylcellulose, methylcellulose, ethylcellulose, hydroxypropylmethylcellulose, acrylic acid co-polymers, carboxymethylcellulose sodium, phthalate, HPMCAS, Eudragit (Rohm Pharma Co., West Germany, acrylate co-polymer, amionic in character), polyvinylacetaldiethylaminoacetate, water soluble salts of enteric coating polymers, and waxes, used alone or in mixtures. Additives, such as plasticizers, colorants, pigments, fillers, anti-tacking and anti-static agents, such as for instance magnesium stearate, titanium dioxide, talc and other additives may also be included into the over-coating layer(s). However, the polymer over-coating does not contain any PPI or other active drug ingredient. The amount of polymer coating in the tablet can range from about 0.01 parts to about 1 part by weight of polymer coating per one part by weight tablet core (about 0.4-3 parts by weight enteric coating:one part tablet core).

The polymer over-coating or tablet film coat can be applied to the enteric coating layered tablet by spraying, coating or layering procedures in suitable equipment, such as coating pan, coating granulator or in a fluidized bed apparatus. In such procedures, water or other solvents may be used to solubilize the materials used for the polymer over-coating or tablet film coat.

The invention can illustrated by the following examples, which are non-limiting as to the scope of the claimed invention.

### Example 1.

Tablet for Delayed Sustained Release

| Tablet core | % w/w |
|---|---|
| Rabeprazole sodium | 10 |
| Lactose F.F. | 76 |
| Sodium starch glycolate | 9 |
| Magnesium stearate | 5 |

The tablet core is prepared by dry mixing rabeprazole sodium with lactose F.F., sodium starch glycolate and magnesium stearate. The dry mixture is compressed with a suitable tablet press into 200 mg core tablets containing 20 mg of rabeprazole sodium which are 0.31″(7.9 mm) in diameter and 0.16″ (4.1 mm) in thickness.

| Tablet core | % w/w |
|---|---|
| Eudragit L100-55 | 49.0 |
| HPMCP HP-55 | 24.5 |
| Lactose F.F. | 24.5 |
| Magnesium stearate | 2.0 |

The enteric coating is prepared by dry blending or mixing Eudragit L100-55, HPMCP HP-55, Lactose F.F. and magnesium stearate. The core tablet is compression-coated using the resulting dry blend to produce 600 mg tablets, 0.40″ (10.2 mm) in diameter and 0.25″ (6.35 mm) in thickness.

The compression-coated tablets are over-coated with 5% hydroxypropylmethylcellulose based on the total tablet weight.

The release of the drug from the tablets is monitored using a dissolution tester, in which 900 mL of simulated gastric fluid (SGF), without enzyme is maintained at 37° C. and used as the dissolution medium for the first 2 hours. The USP 2 dissolution method is used at a rotation speed of 50 rpm. For the next hour, phosphate buffer is used as a media.

Delayed release of rabeprazole sodium is obtained after a period of about 2 hours in SGF dissolution media. The dissolution in phosphate buffer (average, n=6) is as follows:

| Time | % dissolved |
|---|---|
| Media: SGF | |
| 1 hour | 0 |
| 2 hours | 0 |
| Media: phosphate buffer | |
| 5 minutes | 0.9 |
| 10 minutes | 2.0 |
| 15 minutes | 3.5 |
| 30 minutes | 38.7 |
| 45 minutes | 89.4 |
| 60 minutes | 107.5 |

In the following Examples 2-5, the dimensions of the tablet cores and polymer coat are the same as those in Example 1.

### EXAMPLE 2.

Tablet for Delayed Sustained Release

| | % w/w |
|---|---|
| Tablet core | |
| Rabeprazole sodium | 10.0 |
| Lactose F.F. | 76.0 |
| Sodium starch glycolate | 9.0 |
| Magnesium stearate | 5.0 |
| Enteric outer-coating | |
| Eudragit L100-55 | 42.5 |
| HPMCP HP-55 | 31.0 |
| Lactose F.F. | 24.5 |
| Magnesium stearate | 2.0 |

Delayed-release tablets are prepared and tested as for Example 1.

Delayed release of the active ingredient is obtained after a period of about 2 hours in SGF and about 1 hour in phosphate buffer solution.

### Example 3.

Tablet for Delayed Sustained Release

| | % w/w |
|---|---|
| Tablet core | |
| Rabeprazole sodium | 10.00 |
| Lactose F.F. | 76.00 |
| Sodium starch glycolate | 9.00 |
| Magnesium stearate | 5.00 |
| Enteric outer layer | |
| Eudragit L100-55 | 36.75 |
| HPMCP HP-55 | 36.75 |
| Lactose F.F. | 24.50 |
| Magnesium stearate | 2.00 |

Delayed-release tablets are prepared and tested as for Example 1, except that the compression-coated tablets are over-coated with 3% Eudragit polymer based on the total tablet weight.

The release of the drug from the tablets is monitored using a dissolution, in which 900 mL of SGF, without enzyme, is maintained at 37° C. and used as the dissolution medium for the first 2 hours. The USP 2 dissolution method is used at a rotation speed of 50 rpm. For the next hour, phosphate buffer is used as a media.

Delayed release of rabeprazole sodium is obtained after a period of about 2 hours in SGF dissolution media. The dissolution in phosphate buffer (average, n=6) is as follows:

| Time | % dissolved |
|---|---|
| Media: SGF | |
| 2 hours | 0.8 |
| Media: phosphate buffer | |
| 5 minutes | 1.3 |
| 10 minutes | 3.6 |
| 15 minutes | 11.4 |
| 30 minutes | 49.3 |
| 45 minutes | 77.8 |
| 60 minutes | 100.9 |

### EXAMPLE 4.

Tablet for Delayed Sustained Release

| Tablet core | % w/w |
|---|---|
| Rabeprazole sodium | 10 |
| Lactose F.F. | 69 |
| Sodium starch glycolate | 20 |
| Magnesium stearate | 1 |

The tablet core is prepared by dry mixing rabeprazole sodium with lactose F.F., sodium starch glycolate and magnesium stearate. The dry mixture is compressed with a suitable tablet press into 200 mg core tablets containing 20 mg of rabeprazole sodium which are 0.31″ (7.9 mm) in diameter and 0.16″ (4.1 mm) in thickness.

| Enteric outer layer | % w/w |
|---|---|
| Eudragit L100-55 | 49.0 |
| HPMCP HP-55 | 24.5 |
| Lactose F.F. | 24.5 |
| Magnesium stearate | 2.0 |

The enteric outer layer is prepared by dry blending or mixing the above excipients and compression-coating the core tablet with the resulting blend to produce 600 mg tablets, 0.40″ (10.2 mm) in diameter and 0.25″ (6.35 mm) in thickness.

The compression-coated tablets are over-coated with 5% hydroxypropylmethylcellulose based on the total tablet weight.

The release of the drug from the tablets is monitored using a dissolution, in which 900 mL of SGF, without enzyme is maintained at 37° C. and used as the dissolution medium for the first 2 hours. The USP 2 dissolution method is used at a rotation speed of 50 rpm. For the next hour, phosphate buffer is used as a media.

Delayed release of rabeprazole sodium is obtained after a period of about 2 hours in SGF dissolution media. The dissolution in phosphate buffer (average, n=6) is as follows:

| Time | % dissolved |
|---|---|
| Media: SGF | |
| 2 hours | 0.6 |
| Media: phosphate buffer | |
| 5 minutes | 1.3 |
| 10 minutes | 1.9 |
| 15 minutes | 3.3 |
| 30 minutes | 12.6 |
| 45 minutes | 29.8 |
| 60 minutes | 46.7 |

### EXAMPLE 5.

Tablet for Delayed Sustained Release

| | % w/w |
|---|---|
| Tablet core | |
| Rabeprazole sodium | 10.00 |
| Lactose F.F. | 69.00 |
| Sodium starch glycolate | 20.00 |
| Magnesium stearate | 1.00 |
| Enteric outer layer | |
| Eudragit L100-55 | 36.75 |
| HPMCP HP-55 | 36.75 |
| Lactose F.F. | 24.50 |
| Magnesium stearate | 2.00 |

Delayed-release tablets are prepared and tested as for Example 1.

Delayed release of the active ingredient is obtained after a period of about 2 hours in SGF and about 1 hour in phosphate buffer solution.

### EXAMPLE 6.

Tablet for Delayed Sustained Release

| | % w/w |
|---|---|
| Tablet core | |
| Iansoprazole sodium | 15.0 |
| Lactose F.F. | 71.0 |
| Sodium starch glycolate | 9.0 |
| Magnesium stearate | 5.0 |
| Enteric outer layer | |
| Eudragit L100-55 | 24.5 |
| HPMCP HP-55 | 49.0 |
| Lactose F.F. | 24.5 |
| Magnesium stearate | 2.0 |

Delayed-release tablets are prepared and tested as for Example 1.

Delayed release of the active ingredient is obtained after a period of about 2 hours in SGF and about 1 hour in phosphate buffer solution.

### Example 7.

Tablet for Delayed Sustained Release

| | % w/w |
|---|---|
| Tablet core | |
| Pantoprazole sodium | 20.0 |
| Lactose F.F. | 66.0 |
| Sodium starch glycolate | 9.0 |
| Magnesium stearate | 5.0 |
| Enteric outer layer | |
| Eudragit L100-55 | 24.5 |
| HPMCP HP-55 | 49.0 |
| Lactose F.F. | 24.5 |
| Magnesium stearate | 2.0 |

Delayed-release tablets are prepared and tested as for Example 1.

Delayed release of the active ingredient is obtained after a period of about 2 hours in SGF and about 1 hour in phosphate buffer solution.

### Example 8.

Tablet for Delayed Sustained Release

| | % w/w |
|---|---|
| Tablet core | |
| Omeprazole | 10.00 |
| Lactose F.F. | 69.00 |
| Sodium starch glycolate | 20.00 |
| Magnesium stearate | 1.00 |
| Enteric outer layer | |
| Eudragit L100-55 | 36.75 |
| HPMCP HP-55 | 36.75 |
| Lactose F.F. | 24.50 |
| Magnesium stearate | 2.00 |

Delayed-release tablets are prepared and tested as for Example 1.

Delayed release of the active ingredient is obtained after a period of about 2 hours in SGF and about 1 hour in phosphate buffer solution.

### EXAMPLE 9.

Tablet for Delayed Sustained Release

| | % w/w |
|---|---|
| Tablet core | |
| Pariprazole | 10.00 |
| Lactose F.F. | 69.00 |
| Sodium starch glycolate | 20.00 |
| Magnesium stearate | 1.00 |
| Enteric outer layer | |
| Eudragit L100-55 | 36.75 |
| HPMCP HP-55 | 36.75 |
| Lactose F.F. | 24.50 |
| Magnesium stearate | 2.00 |

Delayed-release tablets are prepared and tested as for Example 1.

Delayed release of the active ingredient is obtained after a period of about 2 hours in SGF and about 1 hour in phosphate buffer solution.

### EXAMPLE 10.

Tablet for Delayed Sustained Release

| | % w/w |
|---|---|
| Tablet core | |
| Lemiprazole | 10.00 |
| Lactose F.F. | 69.00 |
| Sodium starch glycolate | 20.00 |
| Magnesium stearate | 1.00 |
| Enteric outer layer | |
| Eudragit L100-55 | 36.75 |
| HPMCP HP-55 | 36.75 |
| Lactose F.F. | 24.50 |
| Magnesium stearate | 2.00 |

Delayed-release tablets are prepared and tested as for Example 1.

Delayed release of the active ingredient is obtained after a period of about 2 hours in SGF and about 1 hour in phosphate buffer solution.

### EXAMPLE 11.

Tablet for Delayed Sustained Release

| | % w/w |
|---|---|
| Tablet core | |
| Esomeprazole | 10.00 |
| Lactose F.F. | 69.00 |
| Sodium starch glycolate | 20.00 |
| Magnesium stearate | 1.00 |
| Enteric outer layer | |
| Eudragit L100-55 | 36.75 |
| HPMCP HP-55 | 36.75 |
| Lactose F.F. | 24.50 |
| Magnesium stearate | 2.00 |

Delayed-release tablets are prepared and tested as for Example 1.

Delayed release of the active ingredient is obtained after a period of about 2 hours in SGF and about 1 hour in phosphate buffer solution.

### EXAMPLE 12.

Development of Delayed Release Tablets Coated with Enteric Polymers by Using Compression Coating Techniques

Purpose. To investigate compression coating as a technique for delayed drug release using enteric polymers. Enteric coating has been typically achieved by using film coating with pH sensitive polymers.

Methods. Drug-containing tablets were coated with enteric coating polymer by using compression coating technique. Delayed release properties, compressibility, and stability were evaluated by applying different compression coating formulations, coating levels, and enteric coating polymer types. Drug release is monitored in vitro in 0.1 N HCI solution for 2 hours and then in pH 7.4 phosphate buffer medium for another 60 minutes using USP apparatus II. Formulation stability is evaluated by comparing compression coating formulation and aqueous enteric coating formulation and by storing at room temperature and at 40° C./75% relative humidity conditions for 3 months. Impurity of the drug in the formulations was determined at initial time, one month, two months, and three months of storage time by HPLC method.

Results. Acid protection is achieved and found to be dependent upon enteric coating levels and compression forces. However, drug release rate in the pH 7.4 phosphate buffer medium are also affected with higher enteric coating level and compression force, which could be ascribed to lower permeability of the compression coating layer. Dissolution results for the optimized formulation show adequate enteric protection and resistance to drug release in 0.1 N HCI while greater than 80% drug is released in pH 7.4 phosphate buffer medium in 60 minutes. This is comparable to enteric polymer coated tablets produced by aqueous coating method. Stability data showed that the total impurity of the formulation could be significantly improved as compared to the aqueous film coating formulation for an acid labile drug. Conclusion. The novel delayed release formulation could be optimized and developed by compression coating with enteric coating polymer formulations. This technique of providing enteric protection is particularly useful where the drug is highly reactive to an aqueous environment during processing.

1. An oral pharmaceutical composition in a solid dosage form comprising:
a) a single core comprising a proton pump inhibitor and a lubricant, wherein said single core has an exterior surface;
b) an enteric compression coating comprising a polymer and a lubricant, wherein said enteric compression coating is on the exterior surface of said single core, without a separating layer between said single core and said enteric compression coating; and
c) optionally, a polymer overcoating on said enteric compression coating.

2. The oral pharmaceutical composition of claim 1 wherein said single core comprises a proton pump inhibitor selected from the group consisting of rabeprazole, omeprazole, esomeprazole, lansoprazole, leminoprazole, pantoprazole or mixtures thereof.

3. The oral pharmaceutical composition of claim 1 wherein said single core comprises a proton pump inhibitor that is rabeprazole.

4. The oral composition of claim 1 containing the sodium salt of rabeprazole.

5. The oral pharmaceutical composition of claim 1 wherein said enteric compression coating comprises a polymer selected from cellulose acetate phthalate, hydroxypropylmethylcellulose acetate succinate, hydroxypropylmethylcellulose phthalate, polyvinylacetate phthalate, carboxymethylethylcellulose, acrylic acid polymers and copolymers and methacrylic acid polymers and copolymers or combinations thereof.

6. The oral pharmaceutical composition of claim 1 in the form of a tablet.

7. An oral pharmaceutical composition in a solid dosage form comprising:
a) a single core comprising a proton pump inhibitor, a disintegrant, a filler and a lubricant, wherein said single core has an exterior surface;
b) an enteric compression coating comprising a polymer and a lubricant, wherein said enteric compression coating is on the exterior surface of said single core, without a separating layer between said single core and said enteric compression coating; and
c) optionally, a polymer overcoating on said enteric compression coating.

8. The oral pharmaceutical composition of claim 7 wherein said single core comprises a proton pump inhibitor selected from the group consisting of rabeprazole, omeprazole, esomeprazole, lansoprazole, leminoprazole, pantoprazole or mixtures thereof.

9. The oral pharmaceutical composition of claim 7 wherein said single core comprises a proton pump inhibitor that is rabeprazole.

10. The oral composition of claim 7 containing the sodium salt of rabeprazole.

11. The oral pharmaceutical composition of claim 7 wherein said enteric compression coating comprises a polymer selected from cellulose acetate phthalate, hydroxypropylmethylcellulose acetate succinate, hydroxypropylmethylcellulose phthalate, polyvinylacetate phthalate, carboxymethylethylcellulose, acrylic acid polymers and copolymers and methacrylic acid polymers and copolymers or combinations thereof.

12. The oral pharmaceutical composition of claim 7 in the form of a tablet.

13. An oral pharmaceutical composition in a solid dosage form comprising:
a) a single core comprising a proton pump inhibitor and a lubricant, said single core being essentially free of an alkaline reacting agent, wherein said single core has an exterior surface;
b) an enteric compression coating comprising a polymer and a lubricant, wherein said enteric compression coating is on the exterior surface of said single core, without a separating layer between said single core and said enteric compression coating; and
c) optionally, a polymer overcoating on said enteric compression coating.

14. The oral pharmaceutical composition of claim 13 wherein said single core comprises a proton pump inhibitor selected from the group consisting of rabeprazole, omeprazole, esomeprazole, lansoprazole, leminoprazole, pantoprazole or mixtures thereof.

15. The oral pharmaceutical composition of claim 13 wherein said single core comprises a proton pump inhibitor that is rabeprazole.

16. The oral composition of claim 13 containing the sodium salt of rabeprazole.

17. The oral pharmaceutical composition of claim 13 wherein said enteric compression coating comprises a polymer selected from cellulose acetate phthalate, hydroxypropylmethylcellulose acetate succinate, hydroxypropylmethylcellulose phthalate, polyvinylacetate phthalate, carboxymethylethylcellulose, acrylic acid polymers and copolymers and methacrylic acid polymers and copolymers or combinations thereof.

18. The oral pharmaceutical composition of claim 13 in the form of a tablet.

19. A process for preparing an oral pharmaceutical composition in a solid dosage form comprising:
a) forming a single core comprising a proton pump inhibitor and a lubricant, wherein said single core has an exterior surface;
b) compression coating an enteric polymer comprising a proton pump inhibitor and a lubricant onto the exterior surface of said single core, in the absence of water and organic solvents, and without forming a separating layer between said single tablet core and said enteric coating; and
c) optionally, applying a polymer overcoating on said enteric compression coating.
